(19) United States
(12) Patent Application Publication
(10) Pub. No.: US 2021/0315930 A1
(43) Pub. Date: Oct. 14, 2021

(54) NUTRACEUTICAL COMPOSITION FOR MULTIMODAL PROPHYLAXIS AGAINST AND TREATMENT OF VIRAL AND BACTERIAL INFECTION AND INFLAMMATION

(71) Applicants: David A. Cuddeback, Binghamton, NY (US); Thomas J. Lynch, Elverson, PA (US)

(72) Inventors: David A. Cuddeback, Binghamton, NY (US); Thomas J. Lynch, Elverson, PA (US)

(21) Appl. No.: 16/848,393

(22) Filed: Apr. 14, 2020

(51) Int. Cl. A61K 33/30; A61K 36/28; A61K 36/537; A61K 36/74; A61K 36/185; A61K 31/375; A61K 31/355; A61K 31/19; A61K 36/82; A61K 31/05; A61K 36/15; A61K 9/00; A61K 45/06; A23L 33/15; A23L 33/16; A23L 33/105; A23L 33/12; A23P 10/30

(52) U.S. Cl. CPC A61K 33/30; A23V 2002/00; A61K 36/537; A61K 36/74; A61K 36/185; A61K 31/375; A61K 31/355; A61K 31/19; A61K 36/82; A61K 31/05; A61K 36/15; A61K 9/0053; A61K 45/06; A23L 33/15; A23L 33/16; A23L 33/105; A23L 33/12; A23P 10/30; A61K 36/28

(57) ABSTRACT

A nutraceutical composition for protection against a wide spectrum of viral and bacterial infections and for treatment of established infection and infectious inflammation. The nutraceutical composition includes a novel combination of vitamin, mineral and herbal or other natural and organic supplements. The composition of supplements may be compounded as a pill, tablet, powder, capsule or liquid be taken orally one or more times per day. Vitamin C and vitamin E along with zinc complexed with pyrithione or citrate are used in conjunction with nutraceuticals provided as immune boosters and antiviral agents along with possible anti-inflammatory herbs or other anti-inflammatory substances.

### BACKGROUND OF THE INVENTION

### Field of the Invention

This invention relates to nutraceutical compositions, products and methods that include multimodal prophylaxis against and treatment of viral and bacterial infections and inflammations, especially in human beings.

### Description of Related Art

Before modern pharmaceuticals, mankind depended on natural substances for medical purposes which included, but were not limited to, relieving pain, enhancing sexual and other performance, enhancing sleep, enhancing bowel function, healing wounds, decreasing respiratory difficulty, stopping blood loss, halting the progress of illness and augmenting general wellbeing. Such natural substances included but were not limited to vegetables, herbs, roots, flowers, tree barks and certain animal organs and tissues. A key target of the use of natural substances was abatement of the combination of pain, swelling and redness in any area of the body, a combination which by the 16^{th} century came to be known as inflammation. At that time and before, inflammation was an important target for treatment with natural substances because it was well recognized that inflammation could progress to worse morbidity or death. Although allergic or purely traumatic injury can also cause inflammation, in the 19^{th} century it was discovered that bacterial infection was a chief and more dangerous cause. In the 20^{th} century, knowledge of the causes of inflammation expanded to include viral and fungal infections.

In our time, natural substances have evolved to include isolated atoms known as minerals, isolated biomolecules known as vitamins and purified extracts from any of the above mentioned, historical sources of natural substances. There is a large variety of such modern natural substances, which in modern pill, tablet, liquid, powder or capsule form are now known as dietary or health supplements, or nutraceuticals. Nutraceuticals are currently used for essentially the same array of purposes as those throughout history, but now also for fortification of the immune system against primary acquisition of infection, i.e. prophylaxis, and for fortification of the immune system's ability to defeat infection once it is established.

Modern antibacterial and antiviral vaccines are generally effective against only the bacterial or viral pathogen from which they were pharmaceutically derived, not a range of bacteria or viruses. A small variety of available supplements is targeted at overall fortification of the immune system, with the intent to prevent or mollify a variety of viral and bacterial infections such that no infection occurs, or its symptoms and physiologic effects are minimal and brief. These supplements go by such labels as Immune Boost, Immune Health and others, and many if not most are composed of a single active ingredient, and none claims both immune fortification and inclusion of a known anti-inflammatory substance.

What is needed is a novel and optimal combination of immune fortifying ingredients, each of which works by a different mechanism to prevent, mollify and treat a wide range of viral and bacterial infections (hereafter termed wide-spectrum anti-infectious), and another ingredient or ingredients which is/are anti-inflammatory, in the event that some degree of infectious inflammation becomes established.

What is needed is a novel and unique combination of nutraceuticals whose wide-spectrum prophylactic effect against infection is compounded by its ability to treat already established infection and infectious inflammation, such that its overall therapeutic effect equals more than the sum of its parts, as does the fortuitous combination of vitamin C+thiamine+hydrocortisone in reversal of sepsis, a severely life-threatening condition which can follow routine infection with a virus or bacterium.

### SUMMARY OF THE INVENTION

A novel combination of vitamin, mineral and herbal or other natural, organic supplements (nutraceuticals) for protection against a wide spectrum of viral and bacterial infections, and for treatment of established infection and infectious inflammation. The composition of supplements is to be compounded as a pill, tablet, powder, capsule or liquid to be taken orally one more times per day, or as parenteral liquid.

Among the possible anti-infectious vitamins to be used are vitamins C and E. The mineral to be used is zinc complexed with citrate, or the amino acid methionine, or other cofactor, in order to increase zinc's bioavailability.

Nutraceuticals provided herein as immune boosters and/or antiviral agents are useful for accelerating wound healing and improved immune function. Among the possible anti-infectious herbs or other organic substances to be used are Echinacea purpura, fulvic acid, ginger, taurine, α-lipoic acid, r-lipoic acid, black elderberry extract, sage, Cat's Claw extract (Uncaria tomentosa) and the omega-3 fatty acids, DHA and EPA.

Among the possible anti-inflammatory herbs or other organic anti-Inflammatory substances to be used are Boswellia serrata gum resin, green tea extract, pycnogenol, resveratrol, curcuminoids and the omega-3 fatty acids, DHA and EPA. Methods of using these compositions of vitamins, minerals, herbs and other organic substances and compositions are also disclosed herein. The amount of each individual supplement substance may be varied significantly.

An object of the invention is to provide compositions of nutraceuticals to fortify the human or companion animal immune system in a way that prevents a wide spectrum of known or new strains of viruses and bacteria from starting a new infection in the host.

Another object of the invention is to provide compositions of nutraceuticals to fortify the human or companion animal immune system in a way that permits nascent viral or bacterial infection, once established, to be treated and defeated before the infection can cause serious and/or widespread systemic symptoms and effects, or to minimize such symptoms to a life non-threatening level.

Another object of the invention is to provide compositions of nutraceuticals that prevent or limit inflammation, especially in the lungs, in which virally or bacterially induced inflammation causes arteriolar and capillary leakage and resultant pulmonary edema, excess mucus secretion, possible severe bronchoconstriction and, in the worst case, acute respiratory distress syndrome (ARDS) with airway collapse, any of which phenomena can compromise pulmonary function to the point of fatal respiratory failure.

Another object of the invention is to prevent or limit development of virally induced secondary bacterial infection/inflammation, or vice versa, especially in the lungs. An overall object of the invention is to help flatten the viral epidemic curve of yearly Influenza, and that of any new viral epidemic. Other features and advantages of the present invention will become apparent to those of skill in the art to consideration of the ensuing description and the appended claims.

### DETAILED DESCRIPTION

For the purposes of this specification, the word “nutraceutical”, refers to a food item, or a part of a food item, that offers medical health benefits, including prevention and/or treatment of disease. More particularly, a nutraceutical is a material that is:
a) a dietary supplement containing a nutritive bioactive compound; or
b) a biologically active process on process material derived from a vitamin, mineral, and herbal/organic supplements plant, fungus, and animal, or a portion thereof; where the precise composition of the biologically active process your unprocessed material may be undetermined.

Examples of a biologically active process material may include a finely chopped, powdered, pure read, or cook material derived from plant or animal tissue, or an extract of plant or animal tissue. The immune boosters and or antiviral agents by first class of nutraceuticals. These agents are useful for accelerating improved immune function; and they include extracts from the cone flowers or herbs of the genus Echinacea and mixtures thereof. Zinc and its bioactive salts such as zinc acetate also act as immune boosters in the treatment of the common cold and viral infections.

A novel and unique combination of vitamin, mineral and herbal/organic supplements (nutraceuticals) in one formulary forming one or more compositions and methods for the purpose of fortifying the innate (cellular) and adaptive (hormonal) components of the immune system against bacterial and viral infection, and their propagation, transmission and effects.

Also included in the formulary compositions are two or more natural, herbal or other organic anti-inflammatory substances for the suppression of tissue inflammation, especially in the lungs. The formulary thereby provides multimodal nutraceutical protection against acquisition of infection and against deleterious effects of inflammation in the lungs. The formulary is available in both oral and parenteral forms.

In a preferred embodiment, the formulary composition contains:
25 mg zinc in any of its pyrithione, citrate, gluconate, picolinate, acetate, sulfate or orotate forms, or with the cofactor methionine.
1000 mg vitamin C
200 mg vitamin E
Two or more of the following anti-infectious nutraceuticals:
1000 mg fulvic acid
800 mg Echinacea purpura
400 Sage extract (Salvia officinalis)
300 mg Cat's Claw extract (Uncaria tomentosa)
200 mg Black Elderberry extract (Sambucus nigra) or 200 mg American Elderberry extract (Sambucus canadensis); and
Two or more of the following anti-inflammatory nutraceuticals:
1000 mg Omega-3 fatty acids, eicosapentanoic acid (EPA) and docosahexanoic acid (DHA).
400 mg Curcumin, including its presence in turmeric powder
300 mg Green Tea extract polyphenols.
200 mg gum resin of Boswellia serrata, containing its most anti-inflammatory moiety, 3-acetyl-11-keto-β-boswellic acid (AKBA)
200 mg Resveratrol
100 mg of Pycnogenol, extract of pine bark (Pinus maritima)

Any of the listed anti-infectious nutraceuticals permutated with any of the listed anti-inflammatory and, in term, combined with the Zinc and Vitamin C or E, will confer five levels of anti-infectious protection, including white blood cell fortification and activation, virucidal-bactericidal activity, enhancement of antibody production, anti-oxidant activity and anti-inflammatory activity. The oral formulary, capsule or other, is enteric-coated to prevent breakdown by gastric acid and permit delivery of unaltered nutraceuticals to the duodenum for absorption.

Although the present invention has been described in detail with particular reference in preferred embodiments thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing disclosure description is for illustrative purposes only and do not any way limit the invention which is defined only by the claims.

1. A nutraceutical composition in capsule or other oral form for preventing or mollifying viral or bacterial infection, and also for preventing or mollifying tissue viral or bacterial inflammation consisting of:
an effective amount of vitamin C;
an effective amount of vitamin E;
an effective amount of zinc
an effective amount of an anti-infectious nutraceutical; and
an effective amount of an anti-inflammatory nutraceutical.

2. The composition of claim 1, wherein an effective amount of anti-infectious nutraceuticals includes an effective amount of two or more of the following:
fulvic acid; Echinacea purpura; sage extract; cats claw extract; black elderberry extract.

3. The composition of claim 1, wherein an effective amount of an anti-inflammatory nutraceutical includes an effective amount of two or more of the following:
omega-3 fatty acids; curcumin, including its presence in turmeric powder; green tea extracts polyphenols; gum resin of Boswellia serata; resveratrol; and Pycnogenol extract of maritime pine bark.

4. The composition of claim 1, wherein an effective amount of anti-infectious nutraceuticals and an effective amount of anti-inflammatory nutraceuticals include:
two or more of the following:
fulvic acid;
Echinacea purpura; sage extract; cats claw extract; black elderberry extract, and
two or more of the following:
omega-3 fatty acids; circumin, including its presence in turmeric powder; green tea extracts polyphenols; gum resin of Boswellia serata; resveratrol; and Pycnogenol® extract of maritime pine bark.

5. The composition of claim 4, wherein
the zinc is in the form of pyrithione, citrate, gluconate, picolinate, sulfate, acetate, or orotate.

6. The composition of claim 4, wherein
zinc with the cofactor of methionine.

7. The composition of claim 1, wherein:
the effective amount of vitamin C is 1000 mg; and
the effective amount of vitamin E is 200 mg.

8. The composition as in claim 5, wherein
effective amount of zinc in any of its pyrithione, citrate, gluconate, picolinate, acetate, sulfate, or other forms is 25 mg.

9. The composition as in claim 5, comprising:
two or more of the following anti-infectious nutraceuticals and effective amounts:
1000 mg of fulvic acid;
800 mg Echinacea purpura; 400 mg sage extract (Salvia officinalis);
300 Mg cat's claw extract (Uncaria tomentosa); and
200 mg black elderberry extract (Sambucus nigra) or 200 mg american elderberry extract (Sambucus canadensis).

10. The composition in claim 9, comprising:
two or more of the following anti-inflammatory nutraceuticals and effective amounts:
1000 mg omega-3 fatty acids; eicosapentanoic acid (EPA) and docosahexanoic acid (DHA);
400 mg curcumin, including its presence in turmeric powder;
300 mg green tea extract polyphenols;
200 mg gum resin of Boswellia serrata, containing its most anti-inflammatory moiety, three-acetyl-11-keto-beta-boswellic acid (AKBA);
200 mg Resveratrol;
100 mg of Pycnogenol®, extract of pine maritime bark (Pinus maritima).

11. A method employing the composition recited in claim 10, to prevent or limit development of virally induced infections and inflammation comprising the steps of:
a) preparing the composition of claim 10 for human consumption;
b) processing composition prepared for human consumption recited in claim 10 in tablet, powder, pill, capsule or liquid to be taken orally; and
c) administering the processed composition recited in claim 10 to a human being periodically.

12. A method for treating a patient having symptoms caused by a viral or bacterial infection and related inflammation comprising the step of administering a nutraceutical composition to the patient, said nutraceutical composition consisting of vitamin C, vitamin E, and zinc in any of its pyrithione, citrate, gluconate, picolinate, sulfate, acetate, or orotate forms and appropriate nutraceutical, components for preventing or reducing viral and bacterial infections and inflammations, wherein an effective amount of anti-infectious nutraceuticals and an effective amount of anti-inflammatory nutraceuticals include two or more of the following:
a) Fulvic acid; Echinacea purpura; Sage extract; Cat's Claw extract; Black Elderberry extract; and
two or more of the following:
b) Omega-3 fatty acids; Curcumin, including its presence in turmeric powder; Green Tea extract polyphenols; gum resin of Boswellia serrata; resveratrol; and Pycnogenol extract of pine bark.

13. The method as in claim 12, wherein, the step of adding a nutraceutical composition administering nutraceutical composition containing zinc with the cofactor of methionine.

14. The method as in claim 13, wherein the step of administering the nutraceutical composition in the effective amount of vitamin C 1000 mg; effective amount of vitamin E is 200 mg; and the effective amount of zinc is 25 mg.

15. The method as in claim 14, wherein the step of administering the effective amount of anti-infectious nutraceuticals in the composition include two or more of the following anti-infectious nutraceuticals:
a) 1000 mg fulvic acid;
b) 800 mg Echinacea purpura:
c) 400 mg Sage extract (Salvia officinalis);
d) 300 mg Cat's Claw extract (Unicaria tomentsa); and
e) 200 mg Black Elderberry extract (Sambucus nigra) or 200 mg American Elderberry extract (Sambucus canadensis).

16. The method as in claim 15, wherein the step of administering the effective amount of anti-inflammatory nutraceuticals include two or more of the following anti-inflammatory nutraceuticals:
a) 1000 mg Omega-3 fatty acids, eicosapentanoic acid (EPA) and docosahexanoic acid (DHA);
b) 400 mg curcumin, including its presence in turmeric powder;
c) 300 mg Green Tea extract polyphenols;
d) 200 mg gum resin of Boswellia serrata, containing its most anti-inflammatory moiety, 3-acetyl-11-keto-beta-boswellic acid (AKBA);
f) 200 mg Resveratrol; and
100 mg of Pycnogenol, extract of pine bark (Pinus maritima).

17. A nutraceutical composition to prevent or limit development of virally induced infections and inflammation comprising:
an effective amount of first and second vitamins;
an effective amount of a mineral;
an effective amount of a nutraceutical compound for preventing or reducing viral and bacterial infections; and
an effective amount of a nutraceutical compound that preventing or reducing viral and bacterial inflammations.

18. The nutraceutical composition as in claim 17, wherein:
said first vitamin is vitamin C and said second vitamin is vitamin E;
said mineral is zinc in any of its pyrithione, citrate, gluconate, picolinate, sulfate, acetate, or orotate forms.

19. The nutraceutical composition as in claim 18, wherein
said effective amount of a nutraceutical compound for preventing or reducing viral and bacterial infections includes two or more of the following anti-infectious nutraceuticals:
fulvic acid; Echinacea purpura; sage extract; cat's claw extract; and black elderberry extract over american elderberry extract.

20. The nutraceutical composition as in claim 19, wherein
said effective amount of said nutritional compound preventing or reducing viral and bacterial inflammations includes two or more of the following anti-inflammatory nutraceuticals:
1) omega-3 fatty acids, eicosapentanoic acid (EPA) and docosahexanoic acid (DHA);
2) curcumin, including its presence in turmeric powder;
3) green tea extract polyphenols;
4) gum resin of Boswellia serrata, containing its most anti-inflammatory moiety, AKBA; 5) Resveratrol: and
6) Pycnogenol® extract of maritime pine bark.
